(19) United States
(12) Patent Application Publication
(10) Pub. No.: US 2009/0093819 A1
(43) Pub. Date: Apr. 9, 2009

(54) ANISOTROPIC SPINAL STABILIZATION ROD

(76) Inventor: ABHIJEET JOSHI, Austin, TX (US)

Correspondence Address: SPRINKLE IP LAW GROUP, 1301 W. 25TH STREET, SUITE 408, AUSTIN, TX, 78705, US

(21) Appl. No.: 11/867,838

(22) Filed: Oct. 5, 2007

(51) Int. Cl. A61B 17/58

(52) U.S. Cl. 606/103

(57) ABSTRACT

Embodiments of the disclosure provide an anisotropic spinal stabilization rod useful for connecting a set of bone fasteners that can anchor a spinal stabilization system onto vertebral bodies. The anisotropic spinal stabilization rod comprises a linear body made of a composite material with fibers selectively oriented in one or more directions to approximate a range of motion of a healthy spine.

### TECHNICAL FIELD

This disclosure relates generally to spinal implants, and more particularly to an implantable spinal stabilization rod with custom anisotropic properties that mimic the biomechanical functions of a healthy spine, useful for both fusion and non-fusion spinal stabilization applications.

### BACKGROUND

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures. A spinal fixation system typically includes corrective spinal instrumentation that is attached to selected vertebra of the spine by screws, hooks, and clamps. The corrective spinal instrumentation may include spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Various types of screws, hooks, and clamps have been used for attaching corrective spinal instrumentation to selected portions of a patient's spine. Examples of pedicle screws and other types of attachments are illustrated in U.S. Pat. Nos. 4,763,644; 4,805,602; 4,887,596; 4,950,269; and 5,129,388. Each of these patents is incorporated by reference as if fully set forth herein.

Often, spinal fixation may include rigid (i.e., in a fusion procedure) support for the affected regions of the spine. Such systems limit movement in the affected regions in virtually all directions (for example, in a fused region). More recently, so called “dynamic” systems have been introduced wherein the implants allow at least some movement of the affected regions in at least some directions, i.e., flexion, extension, lateral, or torsional. While at least some known dynamic spinal implant systems may work for their intended purpose, there is always room for improvement.

### SUMMARY

Embodiments disclosed herein can be used as part of a spinal fusion or non-fusion treatment to stabilize the spine and address the omnipresent back pain problem. Specifically, embodiments of an anisotropic spinal stabilization rod disclosed herein can preserve and/or restore the normal biomechanical functions of a healthy spine. Within this disclosure, the term anisotropic describes a material with physical properties that are different in different directions. A material is isotropic when its mechanical properties remain the same in all directions at a given point, although they may change from point to point. According to embodiments disclosed herein, an anisotropic spinal stabilization rod, in all or in part, is made of a composite material with anisotropic behavior at the macro level. Taking advantage of the anisotropic properties provided by the composite material, embodiments of an implantable biomechanical spinal stabilization system, method, and device disclosed herein can facilitate the stabilization of the spine while maintaining/restoring its mobility. As one skilled in the art can appreciate, embodiments of the anisotropic spinal stabilization rod disclosed herein are not limited to posterior dynamic stabilization of the spine and can be universally applied and adapted for other applications where mimicking a natural biomechanical function is desired.

In one embodiment, an anisotropic spinal stabilization rod comprises at least three distinct parts, which are made of two or more different biocompatible materials. These biocompatible materials may have different physical, chemical, and mechanical properties. In one embodiment, the modulus of elasticity of a first part differs from the modulus of elasticity of a second part by at least an order of magnitude. The higher the modulus the stiffer the part is. In one embodiment, an anisotropic spinal stabilization rod comprises a plurality of cylindrical parts joined in an alternate pattern (e.g., stiff-flexible-stiff, flexible-stiff-flexible, stiff-flexible-stiff-flexible, etc.) about a common axis from end to end. Within this disclosure, a stiff part may be made of any biocompatible materials with a relatively high modulus of elasticity. Examples of suitable materials for a stiff part include stainless steel, titanium, or any biocompatible metal and metal alloy, including non-ferromagnetic alloys, as well as a composite material with fibers oriented parallel and/or perpendicular to a central axis of the stiff part. Fibers are the strongest when a load applied thereto is aligned with their central axis. A flexible part may be made of a composite material comprising a matrix and fibers. The matrix, which can be homogeneous or heterogeneous and which can be made of polymers, metals or ceramics, holds the fibers together. In some embodiments, fibers are oriented in one or more directions (e.g., at an angle, parallel, perpendicular, and/or circumferential to a plane of motion of a spine, etc.).

In some embodiments, an anisotropic spinal stabilization rod comprises a first part made of a first biocompatible material, a second part made of a second biocompatible material and coupled to the first part, and a third part made of a third biocompatible material and coupled to the first part, the second part, or both. The first part, the second part, and the third part together form a straight or substantially straight cylindrical body extending along a longitudinal axis. In some embodiments, the first biocompatible material, the second biocompatible material, the third biocompatible material, or a combination is made of a composite material with fibers selectively oriented in different directions to approximate and mimic a human body's natural response to applied loading. By orienting the fibers in selected directions, different portions of the anisotropic spinal stabilization rod can have varying degrees of desired stiffness against abnormal deformation of the spine while allowing the range of motion of a weakened/damaged spine to be preserved and/or restored. In one embodiment, an anisotropic spinal stabilization rod can be part of a spinal stabilization system. One embodiment of a spinal stabilization system comprises a set of bone fasteners for anchoring the spinal stabilization system onto vertebral bodies and an anisotropic spinal stabilization rod connecting the set of bone fasteners.

Other features, advantages, and objects of the disclosure will be better appreciated and understood when considered in conjunction with the following description and the accompanying drawings.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a simplified graphical representation of a side view showing a portion of a healthy spine;

FIG. 2 depicts a simplified graphical representation of a side view showing a portion of a weakened/damaged spine;

FIG. 3 depicts a simplified graphical representation of a side view showing a portion of a healthy spine and various types of movements of the spine;

FIG. 4 depicts a schematic representation of a spine in various positions;

FIG. 5 depicts a plot diagram illustrating the range of motion of a healthy spine, a destabilized spine, and a stabilized spine, according to some embodiments of the disclosure;

FIG. 6 depicts a plot diagram exemplifying a healthy spine's response to applied loading versus a weakened/damaged spine's response to applied loading;

FIG. 7 depicts a simplified graphical representation of a top view showing a spinal stabilization system installed on vertebral bodies and having a pair of stabilization rods connecting two pairs of bone fasteners, according to some embodiments of the disclosure;

FIG. 8 depicts a schematic representation of an anisotropic spinal stabilization rod, according to one embodiment of the disclosure;

FIGS. 9-12 depict simplified schematic representations of an anisotropic spinal stabilization rod with stiff and flexible cylindrical parts joined about a common axis from end to end in alternate patterns, according to some embodiments of the disclosure;

FIG. 13 depicts a simplified schematic representation of one embodiment of an anisotropic spinal stabilization rod made of a composite material;

FIG. 14 depicts a simplified diagrammatic representation of a cross-sectional view of a portion of one embodiment of a composite material;

FIG. 15 depicts a simplified diagrammatic representation of a cross-sectional view of one embodiment of a composite material having fibers oriented in two directions;

FIG. 16 depicts a simplified diagrammatic representation of a perspective view of one embodiment of a composite material having fibers oriented in one direction;

FIG. 17 depicts a simplified diagrammatic representation of a perspective view of one embodiment of a composite material having fibers oriented in one direction at an angle to the central axial of an anisotropic spinal stabilization rod;

FIG. 18 depicts a simplified diagrammatic representation of a perspective view of one embodiment of a composite material having fibers oriented in two directions: one aligns with the central axial of an anisotropic spinal stabilization rod and one is perpendicular thereto;

FIG. 19 depicts a simplified schematic representation of a perspective view of a one embodiment of a composite material having fibers oriented in two directions;

FIG. 20 depicts a schematic representation of a perspective view of one embodiment of an anisotropic spinal stabilization rod having two stiff parts and a flexible intermediate part, all of which are made of a composite material comprising fibers oriented to mimic how a healthy spine responds to applied loading in various positions;

FIG. 21 depicts a schematic representation of a cross-sectional view of one embodiment of an anisotropic spinal stabilization rod at the microscopic level, the anisotropic spinal stabilization rod having two stiff parts and a flexible intermediate part with fibers oriented to provide increased resistance beyond the neutral zone of spinal movements;

FIG. 22A depicts a schematic representation of a cross-sectional microscopic view of one embodiment of a thin disc that can be used to orient fibers to one or more desired directions;

FIG. 22B depicts a schematic representation of the thin disc of FIG. 22A in use, with fibers coming into the thin disc at one angle and exiting at another, according to one embodiment of the disclosure;

FIG. 23 depicts a schematic representation of fibers helically oriented in a particular direction to mimic a natural biomechanical function, according to one embodiment of the disclosure; and

FIG. 24 depicts a simplified graphical representation of a top view showing a spinal stabilization system installed on vertebral bodies and having a pair of anisotropic spinal stabilization rods connecting two pairs of bone fasteners, according to one embodiment of the disclosure.

### DETAILED DESCRIPTION

The disclosure and the various features and advantageous details thereof are explained more fully with reference to the non-limiting embodiments detailed in the following description. Descriptions of well known starting materials, manufacturing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

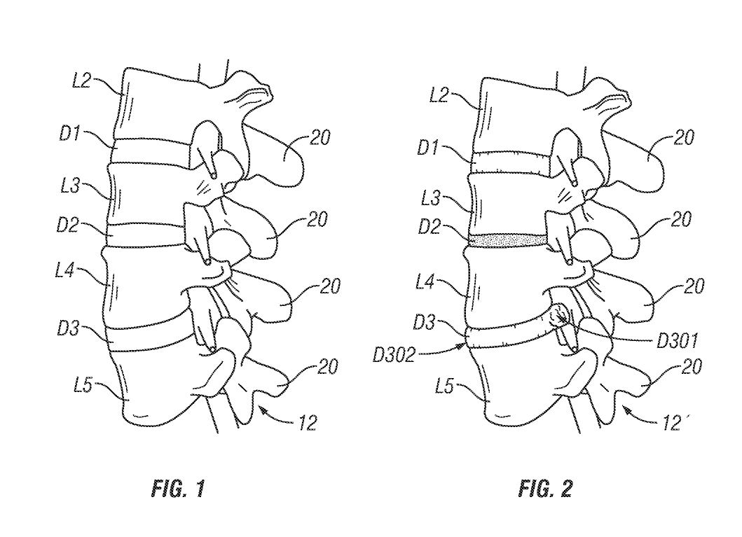

FIG. 1 depicts a simplified graphical representation of a side view of a portion of a healthy spine, which is composed of vertebral bones (i.e., vertebrae) stacked up on one another in a smooth alignment. The spinal canal sits within the spinal column and houses the spinal cord and spinal nerves that send signals to and from the brain. The linkages between the vertebrae are soft, with discs in the front and ligaments in the back, allowing displacement and adaptation to stress and load. Due to the unique and complex arrangements and configurations of vertebrae (e.g., vertebral bodies 20 and vertebral segments L2, L3, L4, L5) and intervertebral disc components, a healthy spine (e.g., spine 12) is strong and flexible and can function through the vigorous demands of daily living, work, and recreational activities. However, the spine is vulnerable to injury and to degeneration, a term which refers to the gradual failure of the spine's biomechanical functions due to aging and wear and tear. The soft tissues (e.g., the intervertebral discs, the ligaments and cartilage of the facet joints) are most vulnerable to degeneration. With normal aging, the discs would gradually collapse and ligaments lose their elasticity and stabilizing ability. Other factors such as those described below may also cause damage to the spine.

FIG. 2 depicts a simplified graphical representation of a side view of a portion of a weakened/damaged spine (e.g., spine 12′). The cause of the damage may be physical (e.g., trauma), natural (e.g., aging/degeneration), disease (e.g., cancer) or a combination thereof. To provide examples, all three discs (D1, D2, and D3) shown in FIG. 2 have abnormalities. Specifically, D1 has a slightly reduced disc height with minimal deterioration, D2 is severely damaged by the degenerative disc disease (DDD), and D3 is a herniated disc with D302 points to a bulging part of D3 and D301 points to the herniation of D3 where a nerve root of the spinal cord is pinched. In the case of DDD, the central nucleus dehydrates and looses its ability to transfer loads to annulus. As is known, disc degeneration leads to disc height loss, altering the normal spinal biomechanics and motion.

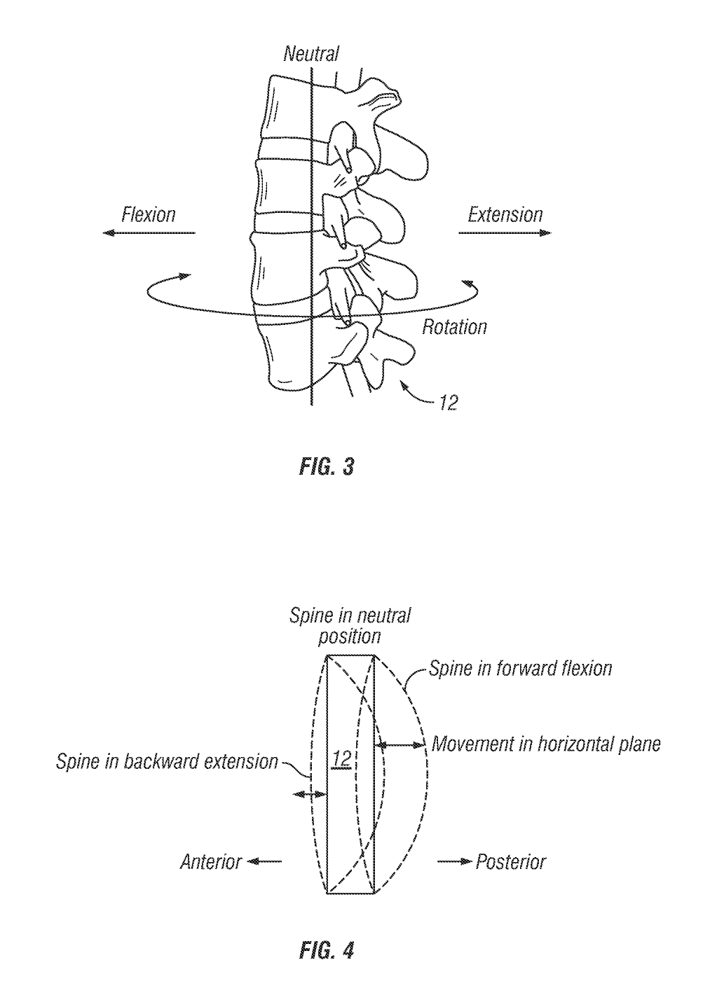

FIG. 3 depicts a simplified graphical representation of a side view of a portion of healthy spine 12 and various types of movements thereof. FIG. 4 depicts a schematic representation of spine 12 in various positions corresponding to the flexion-extension range of motion of the spine in the horizontal plane.

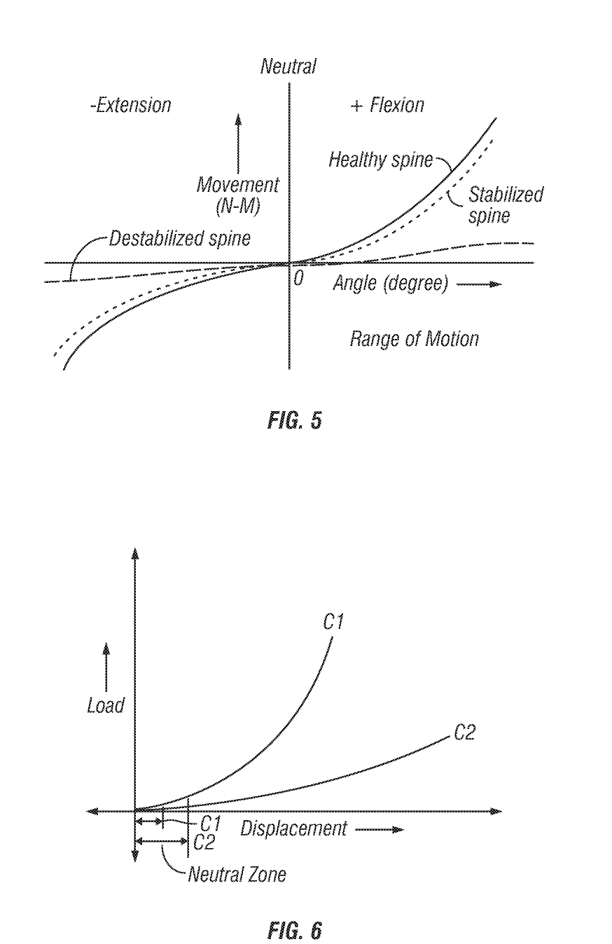

FIG. 5 depicts a plot diagram illustrating the range of motion of a healthy spine, a destabilized spine, and a stabilized spine according to some embodiments of the disclosure. As it can be seen in FIG. 5, when a spine is weakened or destabilized, the range of motion and neutral zone can increase dramatically as compared to a healthy spine.

FIG. 6 depicts a plot diagram exemplifying load-displacement curves of a healthy spine and a weakened/damaged spine. Curve C1 represents the natural response of a healthy spinal unit to applied loading. One example of a spinal unit may be a segment of a spine such as D2, L3, and L4 of FIG. 1. Normally, the spine exhibits non-linear properties as exemplified by Curve C1. That is, when the applied load is low, the spine provides minimal resistance to the applied load and the displacement is high. As the applied load increases, the resistance of the spine to applied load increases and the ratio of displacement to load decreases correspondingly. Curve C2 represents the response of a degenerated or damaged spinal unit to applied loading. When damaged, the mechanical behavior and performance of the spine can change dramatically. As Curve C2 illustrates, the damaged spinal unit is unable to function properly in resisting the applied load and, as the load increases, the ratio of displacement to load continues to increase almost linearly. Comparing Curve C1 and Curve C2, it can be seen that, outside of the neutral zone, a damaged spinal unit would have a much higher displacement-to-load ratio than a healthy spinal unit under the same amount of load, subjecting the annulus and posterior elements to abnormal loading patterns. Many of the spine components have nerve fibers. The abnormal loading patterns can therefore cause debilitation pain. Currently, this pain is treated by a variety of non-surgical (e.g., injection) and surgical approaches.

Surgical approaches to stabilize the spine include spinal fusion and non-fusion treatment options. Spinal fusion, in simplest terms, is a surgical method for removing the damaged intervertebral disc and growing bone structures together to create a solid bone bridge between vertebrae. A fusion of the spine can be done by way of various known methods. The ideal technique in a particular patient will depend upon a number of factors including, but not limited to, the level(s) of vertebrae to be fused, degree of instability or deformity of the spine, age of the patient, risk factors for non-union (i.e., failure to fuse properly), and experience of the surgeon. Known spinal fusions include anterior spinal fusion, posterior spinal fusion with spinal instrumentation, posterior spinal fusion without spinal instrumentation, circumferential fusion (anterior and posterior), posterior lumbar interbody fusion, and transforaminal lumbar interbody fusion. The last two spinal fusion techniques are less invasive than the circumferential fusion and may decrease complications related to open exposures (e.g., infection, wound healing problems, etc.).

Due to the many disadvantages of spinal fusion (e.g., loss of mobility, adjacent disc degeneration due to abnormal load transfer, etc.), non-fusion treatment options may be used in place or in combination with spinal fusion. Non-fusion treatment options are relatively new and may involve replacing and/or stabilizing a damaged portion of the spine with an implant (e.g., a spinal stabilization system, an artificial part such as a spinal disc, etc.).

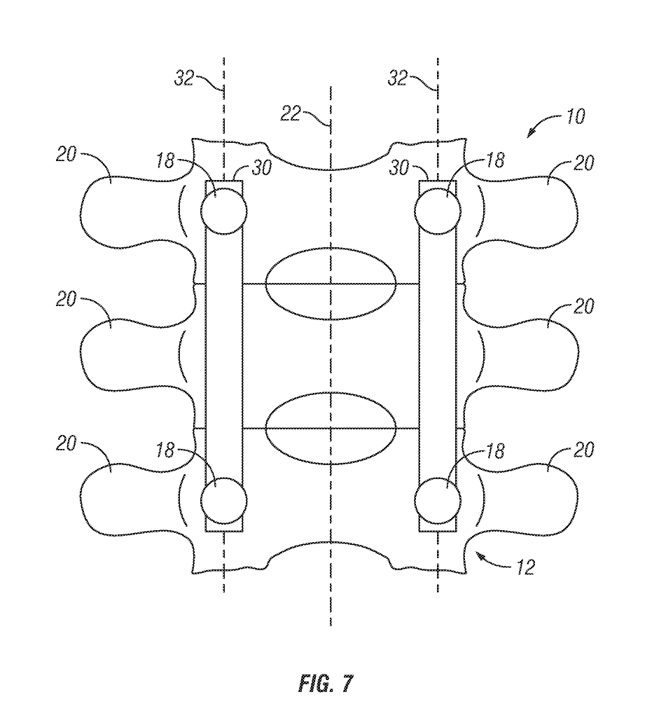

FIG. 7 depicts a simplified graphical representation of a top view showing spinal fixation or stabilization system 10 for supporting spinal column 12, according to some embodiments of the disclosure. In FIG. 7, spinal stabilization system 10 is installed posterior to spine 12 on vertebral bodies 20 and comprises stabilization rods 30 connecting pairs of anchor systems 18. In this example, only one pair of stabilization rods 30 is shown. However, one skilled in the art can appreciate that more than two stabilization rods 30 may be utilized in a spinal procedure (e.g., in a multi-level procedure). Stabilization rods 30 can be fixed laterally on opposite sides of spine 12 to selected vertebra 20 of spine 12, utilizing anchor systems 18. As an example, anchor systems 18 may comprise bone fasteners such as pedicle screws, hooks, claims, wires, etc. Components of spinal stabilization system 10 are made from biocompatible materials. Examples of biocompatible materials include titanium, stainless steel, and any suitable metallic, ceramic, polymeric, and composite materials. Other suitable biocompatible materials are possible and are known to those skilled in the art.

In an un-deformed state, each stabilization rod 30 may extend along a longitudinal axis 32, parallel to the longitudinal axis 22 of the spine 12 lying in the mid-sagittal plane. To stabilize the spine, some embodiments of spinal stabilization system 10 may employ stabilization rods 30 that possess sufficient column strengthen and rigidity to protect the supported portion of spine 12 against lateral forces or movement. One drawback is that the range of spinal motion may be restricted or limited. Table 1 below lists the motions involved with normalized representative values.

TABLE 1

| | Healthy | Destabilized | Stabilized with |
|---|---|---|---|
| Range of Motion | Spine | Spine | Rigid Rod(s) |
| Flexion | 1.0 | 1.40-2.80 | 0.2-0.6 |
| Extension | 1.0 | 1.40-2.20 | 0.2-0.6 |
| Lateral Bending | 1.0 | 1.40-2.40 | 0.2-0.7 |
| Torsion | 1.0 | 1.40-2.40 | 0.2-0.8 |

As is known, the biomechanical functions of a healthy spine are very complex and difficult to replicate. There is a continuing need for better spinal implants and implantable devices that can stabilize a weakened/damaged spine and yet simultaneously allow some range of motion so that the patient can enjoy daily life and normal activities without constraints or restrictions.

In some embodiments, stabilization rods 30 include at least one anisotropic spinal stabilization rod that mimics the biomechanical functions of a healthy spine. As will be described in detail below, such an anisotropic spinal stabilization rod is achieved with a composite material having fibers with controlled, custom anisotropic properties. The term controlled distinguishes composite materials with random anisotropic properties. Customization of the anisotropic properties is accomplished by carefully and selectively aligning the orientation of fibers in the rod with direction(s) in which physiological load(s) might be applied to a patient's spine, particularly to the weakened/damaged spinal unit, during normal daily activities. The orientation of fibers, in addition to other factors such as material selection and production method, allows the anisotropic spinal stabilization rod to carry a load in one direction and yet simultaneously allow bending and/or stretching in another direction(s). As one skilled in the art can appreciate, embodiments of the anisotropic spinal stabilization rod disclosed herein can be useful in both fusion and non-fusion treatment options.

In some embodiments, spinal stabilization system 10 may include additional rods positioned further superior or inferior along spine 12, with the additional rods being anisotropic spinal stabilization rods, dynamic stabilization rods, non-dynamic rods, and/or rigid rods. Within this disclosure, the term “dynamic” refers to the flexing capability of a spinal rod. It should be understood that spinal stabilization system 10 may also include suitable transverse rods or cross-link devices that help protect the supported portion of spine 12 against torsional forces or movement. Some possible examples of suitable cross-link devices are shown in co-pending U.S. patent application Ser. No. 11/234,706, filed on Nov. 23, 2005 and naming Robert J. Jones and Charles R. Forton as inventors (the contents of this application are incorporated fully herein by reference). Other known cross-link devices or transverse rods may also be employed.

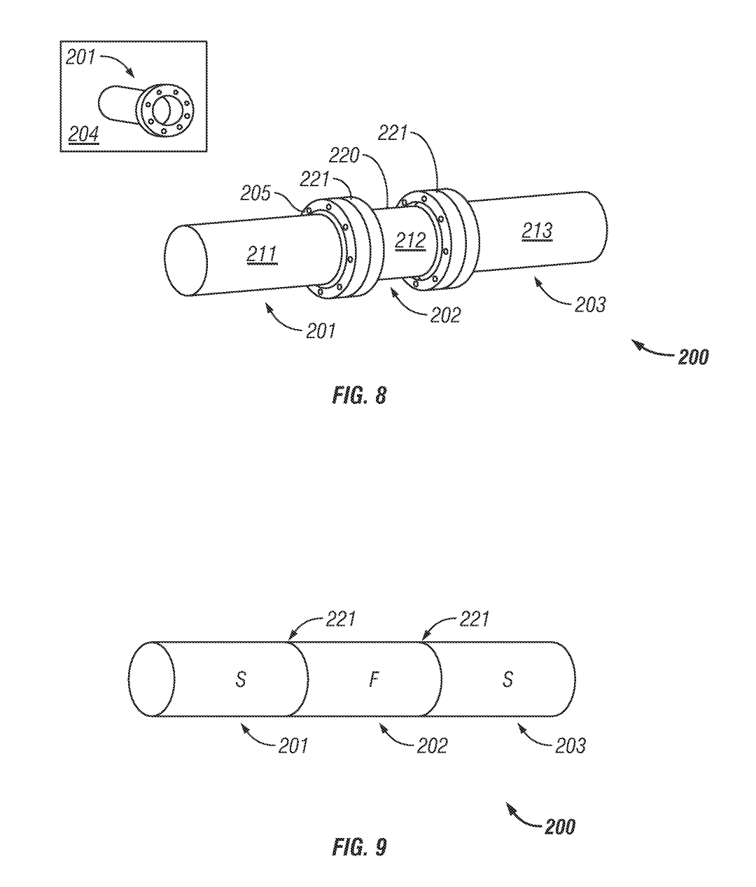

FIG. 8 depicts a schematic representation of anisotropic spinal stabilization rod 200 according to one exemplary embodiment of the disclosure. In this example, anisotropic spinal stabilization rod 200 comprises three distinct cylindrical parts: first part 201 made of first biocompatible material 211, second part 202 made of second biocompatible material 212, and third part 203 made of third biocompatible material 213. As described below with reference to FIGS. 9-12, first part 201, second part 202, third part 203, and possibly an additional part or parts may be arranged in various patterns to form embodiments of anisotropic spinal stabilization rod 200. In one embodiment, part 202 is coupled to part 201 and part 203 from end to end, forming a cylindrical body extending linearly along a longitudinal axis. As depicted in FIGS. 8-13, in the normal, un-deformed state, the cylindrical body of anisotropic spinal stabilization rod 200 can be straight or substantially straight.

In some embodiments, first biocompatible material 211, second biocompatible material 212, third biocompatible material 213, or a combination thereof may be a composite material comprising oriented fibers with custom anisotropic properties that mimic a natural human body response to applied loading. Composites are manipulatable and light weight. They can be as strong and as flexible, depending on the base matrix and fibers properties, fiber content, etc. In the past, fibers are used as reinforcement in composite materials. The flexibility of a composite material is therefore largely dependent on the fiber diameter, the number of layers, and the percentage of fiber volume. Correspondingly, the stiffness of a final product is dependent on the flexibility of the composite material and the geometry of the final product. In embodiments disclosed herein, fiber orientation is the primary factor that affects the flexibility of a composite material. For example, in spinal stabilization applications, anisotropic spinal stabilization rod 200 may have fibers specifically and selectively oriented to support the load in one direction with respect to a plane of motion of a spine while allowing flexibility in another direction or directions to mimic the range of motion of a healthy spine.

In one embodiment, anisotropic spinal stabilization rod 200 may comprise more than three parts, all of which is made of either first biocompatible material 211 or second biocompatible material 212. Depending upon the pattern and/or length desired (see FIGS. 9-12), third biocompatible material 213 may be the same as either first biocompatible material 211 or second biocompatible material 212. In one embodiment, third biocompatible material 213 is the same as first biocompatible material 211 and first biocompatible material 211 differs from second biocompatible 212. In one embodiment, the modulus of elasticity of first part 201 differs from the modulus of elasticity of second part 202 by at least an order of magnitude. In one embodiment, the modulus of elasticity of first part 201 is higher than the modulus of elasticity of second part 202.

In some embodiments, part 201 may be made of any biocompatible materials with a relatively high modulus of elasticity (e.g., stainless steel, titanium, or any biocompatible metal and metal alloy, including non-ferromagnetic alloys, or a composite material with fibers oriented parallel and/or perpendicular to the central axis of part 201). In some embodiments, part 202 may be made of a composite material comprising a matrix and fibers. The matrix, which can be homogeneous (i.e., made from a single material) or heterogeneous (i.e., made from more than one material), holds the fibers together. Any matrix material, including polymers and non-polymer types known or in development, can be utilized. Fibers can be made from a single material; however, the fiber material itself could be derived from more than one material. Examples of suitable materials include glass, carbon, Polyetheretherketone (PEEK), Polyetherketoneketone (PEKK), Ultra high molecular weight polyethylene (UHMWPE) or other artificially derived materials. Other suitable materials are possible and are known to those skilled in the art. Methods of making fibers are also known in the art and thus are not further described herein for the sake of brevity.

In addition to fiber orientation, the final properties of the composite material can depend on a plurality of other factors, including fiber content, fiber material, matrix material, the number of fiber layers in the matrix, manufacturing methods, and so on. In some embodiments, the fiber content of a part is about 10% to 30% by volume. Depending upon the application, a matrix may hold one or more layers of fibers arranged in one or more different directions, including circumferential, with respect to a plane of motion. For example, the fibers of part 202 can have alternate orientation in adjacent layers in one implant, or they can be oriented in alternate orientation in a right and left implant system. Orientation of the fibers can be customized based on the surgeon's recommendation. For example, if the patient needs stability on left torsion, then the fibers can be oriented in such a way so as to allow a spinal stabilization system (e.g., spinal stabilization system 10) when implanted properly to resist left torsion after certain extent. More details on fibers with different orientation arrangements and examples thereof are described below with reference to FIGS. 19-23.

Referring to FIG. 8, anisotropic spinal stabilization rod 200 can share load and allow the instrumented spinal segment to approximate the physiological motion in flexion-extension. In addition, anisotropic spinal stabilization rod 200 can provide stability in lateral bending, axial rotation and in shear while allowing motions that mimic the natural, non-linear response of a healthy spine. As one skilled in the art can appreciate, the geometry of anisotropic spinal stabilization rod 200 can vary according to the need and the operating level (e.g., thoracic, lumbar, etc.). Likewise, part 201, part 202, and part 203 can be joined in many ways. In some embodiments, part 201, part 202, and part 203 can be bolted together, using holes 205, which may be threaded, and screws (not shown) at mating ends. Exploded view 204 depicts a mating end of part 201. Bolting part 201, part 202, and part 203 together can create a solid junction at joints 221 while allowing motion and stability in the desired planes. In some embodiments, parts 201 and 203 may have holes along its length (not shown) and part 202 may be sutured through these holes.

The modulus of elasticity of parts 201 and 203 may be the same or substantially the same, depending upon the needs of the patient. Accordingly, in one embodiment, a method of making an anisotropic spinal stabilization rod may comprise forming at least one first part from a first biocompatible material with high modulus of elasticity (e.g., part 201), forming at least one second part from a second biocompatible material comprising a matrix and fibers with controlled anisotropic properties that mimic a range of motion of a healthy spine (e.g., part 202), and then securing (e.g., bolting, press-fitting, suturing, etc.) at least one first part and at least one second part together in an alternating pattern along a longitudinal axis. In one embodiment, the modulus of elasticity of the first biocompatible material is higher (i.e., stiffer) than that of the second biocompatible material by at least an order of magnitude. In one embodiment, the method may further comprise orienting the fibers in one direction. In one embodiment, the method may further comprise orienting the fibers in a first (e.g., vertical) direction. In one embodiment, the method may further comprise orienting the fibers in two or more directions. In one embodiment, the method may further comprise orienting a first layer and a second layer of fibers at an angle (e.g., ±45°, etc.) with respect to a horizontal plane. In one embodiment, the method may further comprise orienting a third layer of fibers perpendicular to the horizontal plane. In some embodiments, the preferred range of angle is about ±28° to 57° with respect to the horizontal plane. In some embodiments, in producing an embodiment of anisotropic spinal stabilization rod 200 or a portion thereof, a single strand of fiber can be oriented to different angles more than once (see e.g., FIGS. 21 and 23).

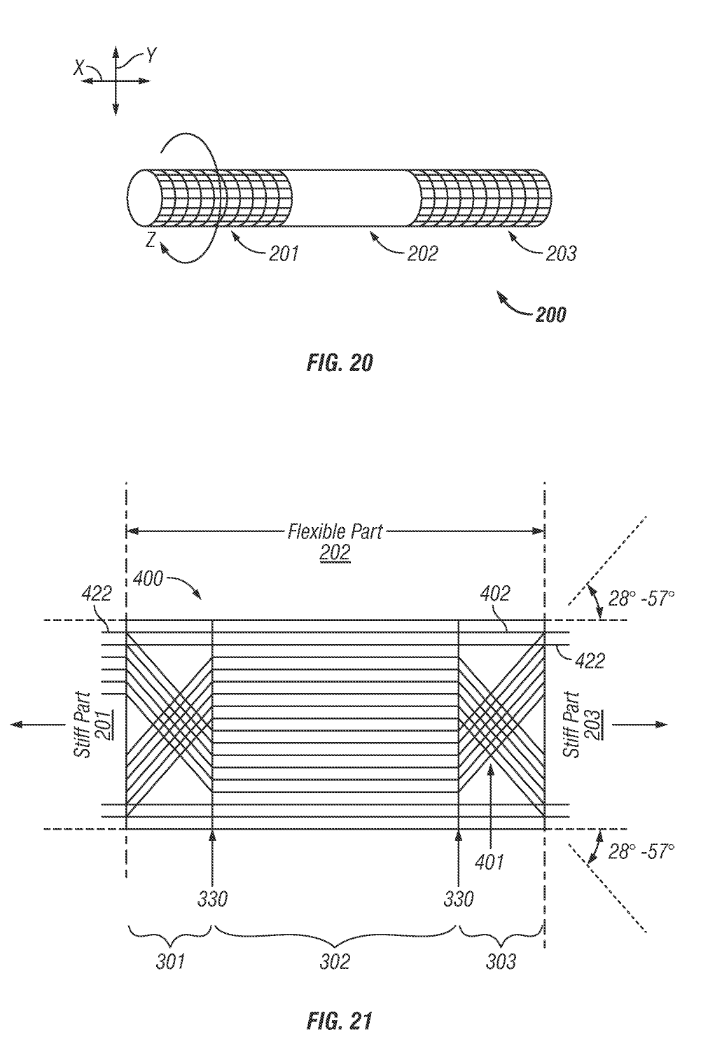

In some embodiments, parts 201, 202, and/or 203 may be hollow inside (e.g., cannulated). In some embodiments, parts 201, part 202, and part 203 may have accommodating inner and outer diameters that allow them to be press-fitted together. FIG. 9 depicts a simplified schematic representation of anisotropic spinal stabilization rod 200 with cylindrical parts 201, 202, and 203 that are joined (e.g., press-fitted) together about a common axis from end-to-end at joints 221. Parts 201, 202, and 203 can be made of different biocompatible materials and arranged in an alternate pattern as shown in FIG. 9. In one embodiment, parts 201 and 203 are made of a stiff material (S) and part 202 is made of a flexible material (F). In one embodiment, the flexible material is a composite with fibers oriented in one or more directions with respect to a plane of motion of a spine. In one embodiment, the flexible material is a composite with fibers oriented in one or more directions with respect to a horizontal plane. In one embodiment, both the stiff and flexible materials are composite materials with oriented fibers (see e.g., FIGS. 20-21). In one embodiment, the modulus of elasticity of part 201 is higher than the modulus of elasticity of part 202 by at least one order of magnitude.

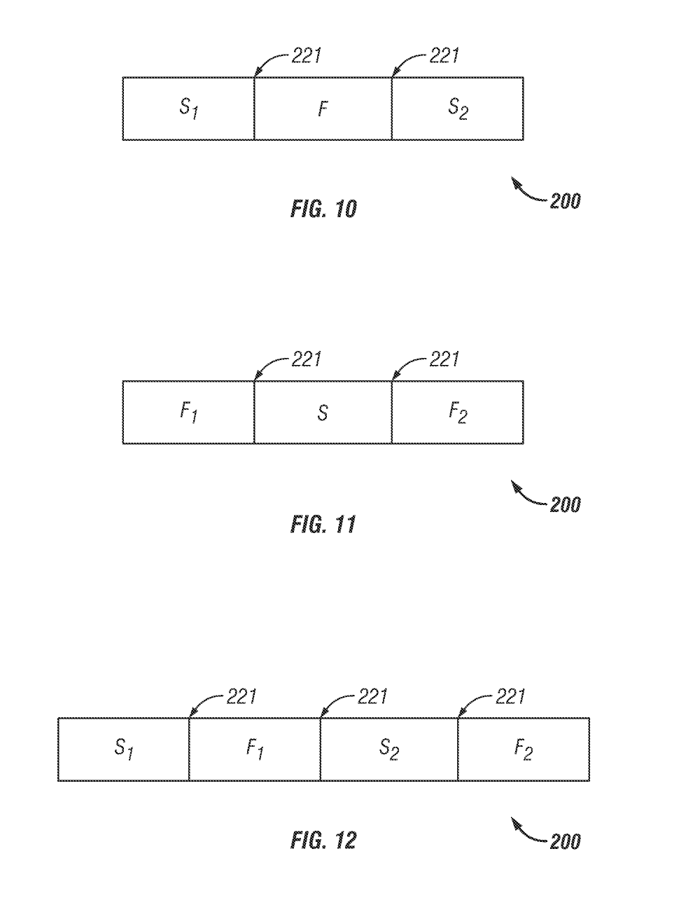

FIGS. 10-12 depict simplified schematic representations of anisotropic spinal stabilization rod 200 with stiff parts (S, S1, and S2) and flexible parts (F, F1, and F2) arranged in alternate patterns, according to some embodiments of the disclosure. These parts have cylindrical bodies and are joined (e.g., bolted, sutured, press-fitted, etc.) together about a common axis from end-to-end at joints 221. S. S1, and S2 can be made of the same or different biocompatible materials with the same or similar modulus of elasticity. F, F1, and F2 can be made of the same or different biocompatible materials with the same or similar modulus of elasticity. In one embodiment, S, S1, and S2 are made of metals or metal alloys and F, F1, and F2 are made of composites with fibers oriented in one or more directions. In one embodiment, F, F1, F2, S, S1, and S2 are composite materials with fibers oriented in one or more directions. In one embodiment, the modulus of elasticity of parts S, S1, and S2 is higher than the modulus of elasticity of parts F, F1, F2 by at least an order of magnitude.

In one embodiment, all parts of anisotropic spinal stabilization rod 200 are of equal or substantially similar size. In one embodiment, the length of each part may be the same or substantially the same. In one embodiment, the length of each part may vary. In one embodiment, one or both ends (e.g., parts 201 and/or 203) of anisotropic spinal stabilization rod 200 can extend to more than one level, making anisotropic spinal stabilization rod 200 useful in a multi-level procedure. In one embodiment, the length of first stiff part (e.g., part 201) might be double than that of the stiff part on the other side of middle flexible part (e.g., part 203).

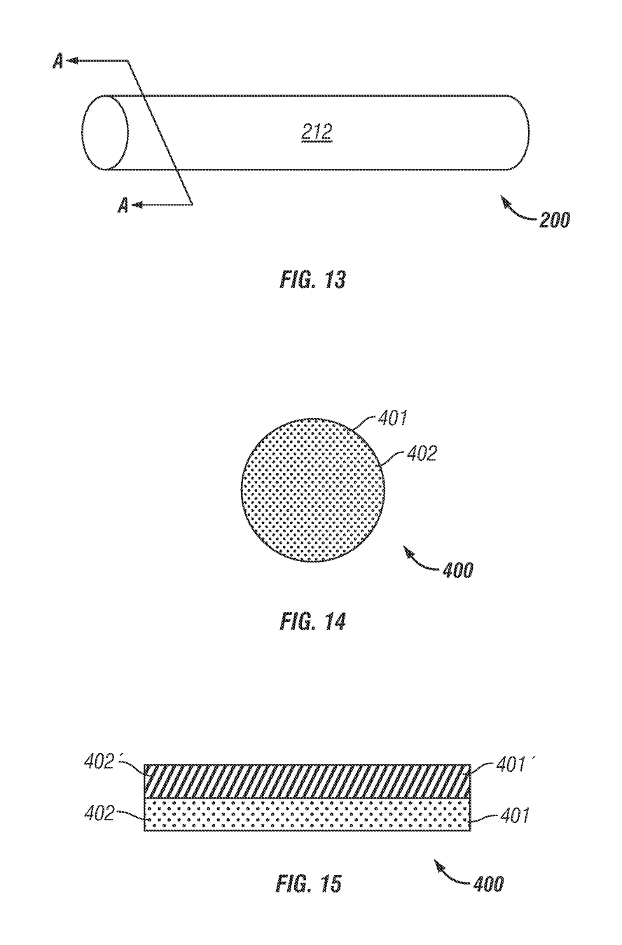

In one embodiment, there are no stiff parts at either or both ends of anisotropic spinal stabilization rod 200. In one embodiment, the entire anisotropic spinal stabilization rod 200 can be made of a composite material, even the part that is for attaching to pedicle screws. FIG. 13 depicts a simplified schematic representation of one embodiment of anisotropic spinal stabilization rod 200 made of composite 212. FIG. 14 depicts a simplified diagrammatic representation of a cross-sectional view of portion 400 of one embodiment of composite 212 taken alone line A-A of FIG. 13. As will be described below with reference to FIGS. 15-21, composite 212 may comprise fibers oriented in different directions (e.g., with respect to a plane of motion or a horizontal plane). Thus, different portions or parts of spinal stabilization rod 200 may have different cross-sectional views. Accordingly, portion 400 of FIG. 14 is representative of composite 212 alone line A-A of FIG. 13 only and not the entire anisotropic spinal stabilization rod 200.

Although anisotropic spinal stabilization rod 200 of FIG. 13 may look like an ordinary rod, it possesses unique and advantageous mechanical and physical properties. As is known, the biomechanical functions of a healthy spine are very difficult to replicate. Ordinary rods themselves cannot mimic or restore the natural response of a healthy spine, even if they are reinforced with fibers. For example, U.S. Pat. No. 4,743,260, issued to Burton, discloses constructing a rod-like vertebral column stabilization element with a two-phase biocompatible plastic reinforced with carbon fibers. In this case, adding fibers solves a practical fabrication problem as the internal diameter of the stabilization element decreases. However, as discussed in U.S. Pat. No. 5,415,661, issued to Holmes, the device disclosed in Burton is disadvantageous because it removes the posterior elements (facet capsules) which provide about 20% of the support inherent of the spine as well as torsional stability for the joint. According to Holmes, linear bar-like elements such as the carbon fiber reinforced plastic element disclosed by Burton cannot provide support and movement which closely approximates the function of the spine.

Instead of linear bar-like elements, Holmes proposes a curvilinear compliant implantable device for restoring normal biomechanical function to a motion segment unit of the spine. The compliant implantable device of Holmes has a flexible curvilinear body composed of a composite material and two terminal sections for attaching the compliant implantable device to adjacent motion segment units. The curved portion of the curvilinear body is free to move in response to pressure applied to a diseased or damaged motion segment unit to provide support for and restore normal biomechanical function to the diseased or damaged motion segment unit. The compliant implantable device of Holmes is said to be able to restore normal motion between the vertebrae and the surrounding motion segment units by supporting and distributing a percentage of the load normally carried by the affected motion segment unit to the surrounding motion segment units. However, distributing or transferring the load to the surrounding motion segment units is undesirable as continuously subjecting adjacent discs with additional load may cause disc degeneration in the long run.

Embodiments of an anisotropic spinal stabilization rod disclosed herein can restore/mimic the biomechanical functions of a spine without the aforementioned disadvantages. As exemplified in Table 2 below, embodiments of an anisotropic spinal stabilization rod disclosed herein can support complex movements of a healthy spine. Like Table 1, values in Table 2 are normalized.

TABLE 2

| | Healthy | Destabilized | Stabilized with Anisotropic |
|---|---|---|---|
| Range of Motion | Spine | Spine | Spinal Stabilization Rod(s) |
| Flexion | 1.0 | 1.40-2.80 | At least 80% of normal spine |
| | | | or ±20% of normal spine |
| Extension | 1.0 | 1.40-2.20 | At least 80% of normal spine |
| | | | or ±20% of normal spine |
| Lateral Bending | 1.0 | 1.40-2.40 | At least 80% of normal spine |
| | | | or ±20% of normal spine |
| Torsion | 1.0 | 1.40-2.40 | At least 80% of normal spine |
| | | | or ±20% of normal spine |

Depending upon implementation, each embodiment of an anisotropic spinal stabilization rod can have selected directional stiffness/flexibility in one or more parts/portions thereof. The stabilized range of motion (see Table 2) is achieved by carefully manipulating, in design and manufacturing, the orientation of each fiber in a composite material of which each anisotropic spinal stabilization rod is made. More specifically, the orientation of the fibers is aligned with each (physiological) load direction so that the rod itself can carry the load in that direction while allowing flexibility in the other direction(s). In this way, in the neutral zone (see FIGS. 3-6), the overall strength of an anisotropic spinal stabilization rod sufficiently provides an initial low resistance to applied load/deformation. Beyond the neutral zone, the composite material of the rod, particularly the orientation of the fibers, enables the rod to carry the load and to provide non-linear support against further deformation—similar to that of a natural healthy spine.

To this extent, in some embodiments, composite material 400 of FIG. 14 comprises base matrix 401 and fibers 402. In FIG. 14, fibers 402 are perpendicular to the plane of paper. In the example shown in FIG. 13, fibers 402 at line A-A would be parallel lengthwise to the central axis of anisotropic spinal stabilization rod 200.

FIG. 15 depicts a simplified diagrammatic representation of a cross-sectional view of one embodiment composite 400 with fibers oriented in two directions. In the example shown in FIG. 15, the bottom layer has fibers 402 oriented in matrix 401 in one direction in the same manner as described above with reference to FIG. 14. The top layer of composite 400 comprises matrix 401′ and fibers 402′, which are shown at an angle. Matrix 401 and matrix 401′ can be homogeneous (i.e., having a uniform composition or structure) or heterogeneous (i.e., having a composition or structure made of individual elements). In embodiments disclosed herein, fibers are the strongest when the load is aligned with their central axis. Thus, to provide desired load carrying strength in a particular direction, for instance, perpendicular to the plane of paper as depicted in FIG. 14, fibers 402 are oriented to align with that direction. In some embodiments, the orientation of fibers can be repeated per layer and each layer of fibers may be oriented in a different direction.

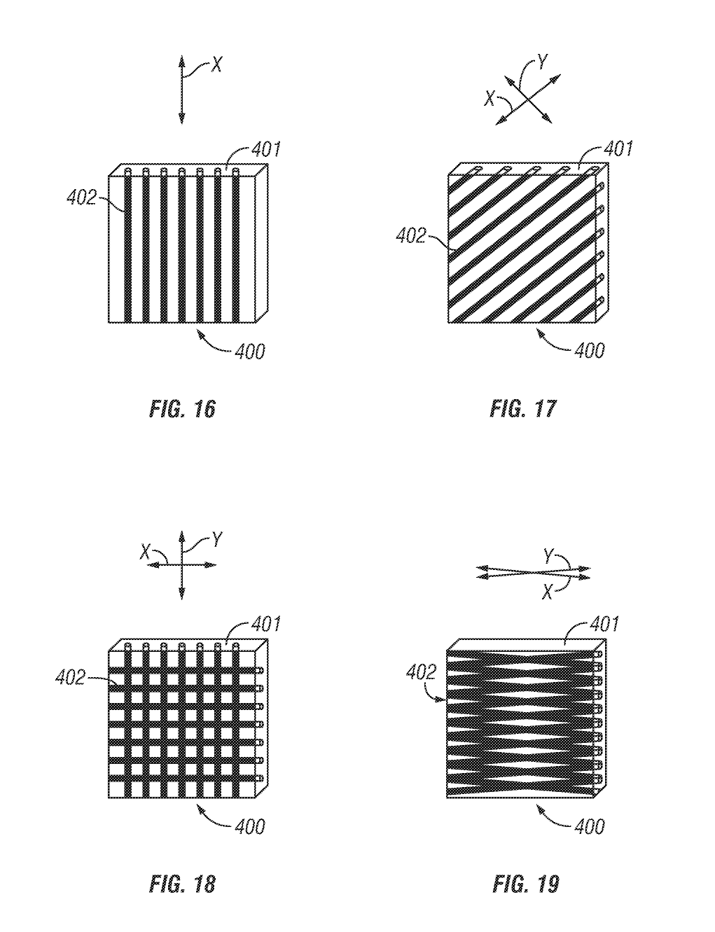

FIG. 16 depicts a simplified diagrammatic representation of a perspective view of one embodiment of composite material 400. In the example of FIG. 16, composite 400 is composed of matrix 401 and fibers 402, with fibers 402 oriented to align with axis X. In this case, fibers 402 of FIG. 16 are the strongest when the load is aligned with axis X. Correspondingly, the load carrying strength of composite 400 is also the strongest in the direction of X. In some embodiments, the configuration of FIG. 16 can be utilized to make a stiff part of anisotropic spinal stabilization rod 200, with axis X being the central or common axis of anisotropic spinal stabilization rod 200.

FIG. 17 depicts a simplified diagrammatic representation of a perspective view of one embodiment of composite material 400 with fibers oriented in one direction X at an angle to the central axis of anisotropic spinal stabilization rod 200 (not shown). As described above, depending on the alignment of fibers 402 within base matrix 401, composite 400 may offer resistance in different directions. For example, the lamina (layer) of FIG. 17 would be stronger (i.e., offers much more resistance) and tougher in the direction indicated by the arrow along fiber axis X. However, in other directions (e.g., axis Y), the lamina is weaker and compressible/flexible because properties of matrix 401 are dominant in that direction as compared to that of fibers 402. This is particularly the case in the direction perpendicular to fiber axis X.

FIG. 18 depicts a simplified diagrammatic representation of a perspective view of one embodiment of composite material 400 with fibers oriented in two directions, one (e.g., axis Y) aligns with the central axis of anisotropic spinal stabilization rod 200 (not shown) and one (e.g., axis X) is perpendicular thereto. In this case, composite 400 is about equally strong in the directions indicated by arrows along axis X and axis Y. In some embodiments, the configuration of FIG. 18 can be utilized to make a stiff part of anisotropic spinal stabilization rod 200.

FIG. 19 depicts a simplified schematic representation of a perspective view of one embodiment of composite material 400 having matrix 401 and at least two layers of fibers 402. Fibers 402 on a first layer may be oriented along axis X and fibers 402 on a second layer may be oriented along axis Y, at an angle from axis X as shown in FIG. 19.

FIG. 20 depicts a schematic representation of a perspective view of one embodiment of anisotropic spinal stabilization rod 200 with stiff parts 201 and 203 and flexible intermediate part 202, all of which are made of a composite material comprising fibers selectively oriented to mimic how a healthy spine responds to applied loading in various positions. In one embodiment, stiff parts 201 and 203 have fibers oriented in three directions: axial direction X, axial direction Y, and circumferential direction Z. The fiber orientation of flexible intermediate part 202 is more complex and will be described below with reference to FIG. 21.

FIG. 21 depicts a schematic representation of a cross-sectional view of one embodiment of anisotropic spinal stabilization rod 200. Like the example shown in FIG. 20, anisotropic spinal stabilization rod 200 has stiff parts 201 and 203 and flexible intermediate part 202 made of composite material 400. Composite 400 has base matrix 401 and fibers 402 oriented to provide increased resistance beyond the neutral zone of spinal movements. In the example shown in FIG. 21, fibers 402 have fiber ends 422 for attachment (e.g., suturing, etc.) to stiff parts 201 and 203. For the sake of clarity, not all fiber ends are shown in FIG. 21. Part 202 can be seen in FIG. 21 as having portions 301, 302, and 303. In this embodiment, fibers 402 in portion 302 are aligned with the common axis of parts 201, 202, and 203. Fibers oriented perpendicular to the plane of motion can provide resistance to vertical extension/stretch after the initial lag. As illustrated in the example of FIG. 21, fibers can be oriented at different angles toward different directions in portions 301, 302, and 303. In this case, upon entering stiff parts 201 and 203, fibers 402 are oriented to be lengthwise parallel to the longitudinal axis of anisotropic spinal stabilization rod 200. Although not shown, fibers 402 may also be oriented circumferentially in parts 201, 202, and/or 203.

In some embodiments, the preferred range of fiber angle is about ±28° to 57°, depending upon application. For example, to approximate the flexion-extension range of motion (see FIG. 4), in the example shown in FIG. 21, fibers 402 are oriented ±45° in portions 301 and 303 with respect to a horizontal plane, which, in this case, is perpendicular to the longitudinal axis of the cylindrical body of rod 200, representing the optimum angle for stabilizing flexion (+45°) and extension (−45°) of the spine.

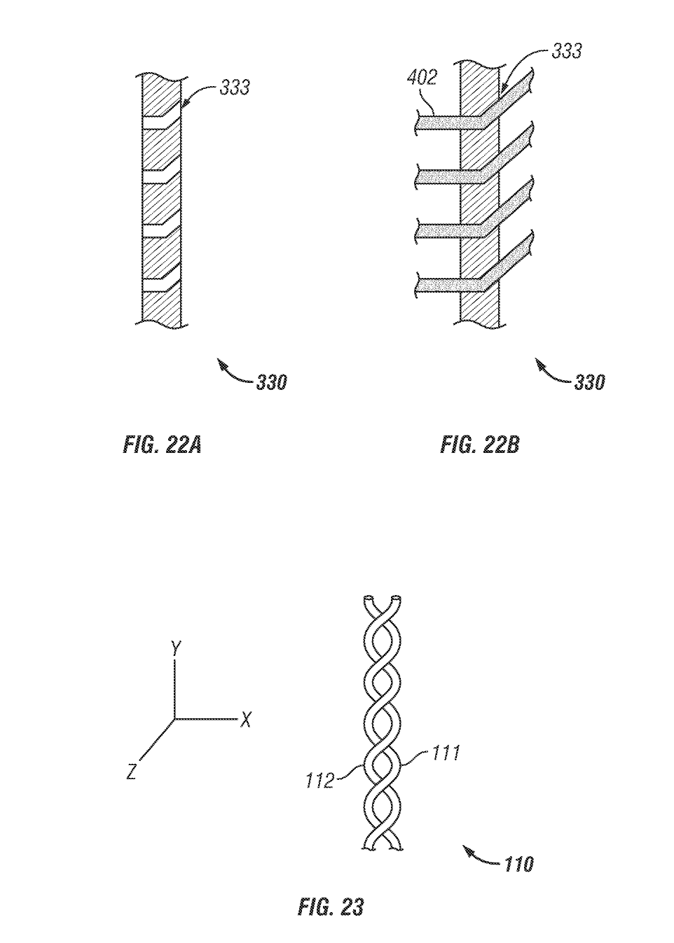

In some embodiments, perforated thin disc(s) 330 may be utilized to facilitate orienting fibers 402 to desired direction(s). FIG. 22A depicts a schematic representation of a cross-sectional microscopic view of one embodiment of thin disc 330 that can be used to orient fibers 402 to one or more desired direction in anisotropic spinal stabilization rod 200. As illustrated in FIG. 22A, in one embodiment, thin disc 330 has a plurality of through holes 333, each of which turns at an angle at about half the depth of thin disc 330. FIG. 22B depicts a schematic representation of thin disc 330 in use, with fibers 402 coming into through holes 333 of thin disc 330 at one angle (e.g., perpendicular to thin disc 330) and exiting at another (e.g., at a 45 degree angle to thin disc 330).

In addition to the examples above, other fiber orientations are also possible. For example, to add torsional stability, one embodiment of an anisotropic spinal stabilization rod may comprise fibers helically aligned in the vertical direction of a Cartesian coordinate system, similar to the DNA structure. FIG. 23 depicts a schematic representation of one embodiment of arrangement 110 having fibers 111 and 112 helically oriented in the direction of axis Y to mimic the resistance of a healthy spine to lateral bending and torsion. In one embodiment, arrangement 110 can allow initial torsional motion/rotation to some extent (e.g., ±3°, total 6 degrees of motion) and resist further instability/excessive rotation. In this way, an anisotropic spinal stabilization rod particularly configured and manufactured with arrangement 110 can stabilize the excessive range of motion in a destabilized spine in lateral bending and torsion.

Embodiments of an anisotropic spinal stabilization rod disclosed herein can be implemented using standard manufacturing methods known to those skilled in the art (e.g., pultrusion, filament winding, electrospinning, 3-D weaving, injection molding, co-curing technique, etc.). For the sake of brevity, suitable starting materials and manufacturing methods are not further described herein. In some embodiments, finite element simulations and other simulation software available in the market (e.g., CADPRESS, MOLDFLOW, etc.) can be used to analyze fiber orientation (e.g., parallel, perpendicular, or at an angle to a plane of motion) with respect to the intended purpose(s) of an anisotropic spinal stabilization rod. In some embodiments, orientation arrangements of the fibers can be done in layers. Such a simulation may therefore include parameters such as fiber content (e.g., about 10% to 30%), the number of fiber layers, the number of fibers in a braid, matrix composition (i.e., homogeneous or heterogeneous), etc. By optimizing the design and manipulating its performance, the time required for a prototype stage or stages can be eliminated or reduced. In particular, by manipulating the fiber alignment with respect to human anatomy, desired range of motion, and other biomechanical functions, the stabilized range of motion set forth in Table 2 above can be achieved.

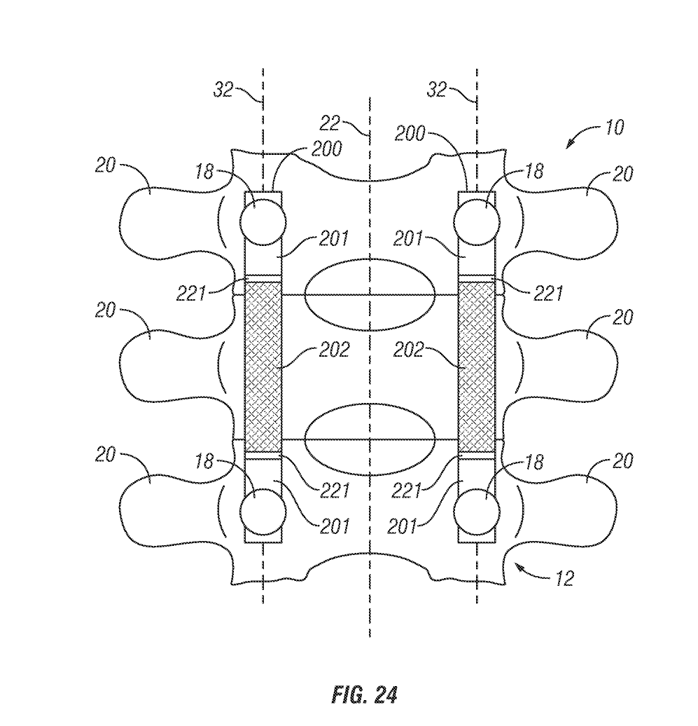

FIG. 24 depicts a simplified graphical representation of a top view of spinal stabilization system 10 installed on vertebral bodies 20 of spine 12. In this example, spinal stabilization system 10 comprises anisotropic spinal stabilization rods 200 for connecting bone fasteners 18 to stabilize spine 12 and for providing support which mimics the range of motion of a healthy spine as described above. In this embodiment, anisotropic spinal stabilization rods 200 is made of stiff parts 201 and flexible part 202 similar to those described above with reference to FIGS. 8-9. Persons skilled in the art may make various changes in the shape, size, number, and/or arrangement of parts without departing from the scope of the disclosure as described herein. To this extent, it should be appreciated that components of spinal stabilization system 10 shown in FIG. 24 are for purposes of illustration only and may be changed as required to render spinal stabilization system 10 suitable for its intended purpose.

Embodiments of spinal stabilization system 10 can provide many advantages. For example, when properly installed, spinal stabilization system 10 may minimize torsional and shear stresses that tend to delaminate an intervertebral disc and protect the level above the operated segment from additional stresses, which might accelerate the adjacent level disc degeneration. Particularly, as anisotropic spinal stabilization rods 200 can unload or partially unload a weakened/damaged disc, mimic the non-linear properties of a healthy spine, and share the load applied thereupon accordingly, spinal stabilization system 10 can advantageously avoid having additional load undesirably transferred to adjacent vertebrae, providing pain relief and restricting any abnormal motion while allowing movement in flexion-extension and stability in lateral bending, torsion, and shear.

Furthermore, spinal stabilization system 10 can intervene earlier in the degeneration cascade and allow the spine to assume an appropriate sagittal balance in a variety of postures. Accordingly, embodiments of spinal stabilization system 10 may find applications in both spinal fusion and non-fusion indications. In fusion, some embodiments of spinal stabilization system 10 may adjunct to fusion in the treatment of the acute and chronic instabilities of the cervical, thoracic, lumbar, and sacral spine with anterior column support such as Degenerative Disc Disease, Degenerative Spondylolisthesis with objective evidence of neurological impairment, fracture, dislocation, deformities, or curvature (e.g., Scoliosis, Kyphosis, disc height change, etc.). In non-fusion, some embodiments of spinal stabilization system 10 may be installed for the dynamic stabilization of the cervical, thoracic or lumbar disc in patients with early disc degeneration.

Another advantage of spinal stabilization system 10 is its versatility. Embodiments of spinal stabilization system 10 may be used in minimally invasive surgery (MIS) procedures as well as non-MIS procedures. It is believed that the ability to implant spinal stabilization system 10 using MIS procedures can provide additional advantages. MIS procedures seek to reduce cutting, bleeding, and tissue damage or disturbance associated with implanting a spinal implant in a patient's body. Exemplary procedures may use a percutaneous technique for implanting longitudinal rods and coupling elements. Examples of MIS procedures and related apparatus are provided in U.S. patent application Ser. No. 10/698,049, filed Oct. 30, 2003, U.S. patent application Ser. No. 10/698,010, filed Oct. 30, 2003, and U.S. patent application Ser. No. 10/697,793, filed Oct. 30, 2003, incorporated herein by reference.

In the foregoing specification, specific embodiments have been described with reference to the accompanying drawings. However, as one skilled in the art can appreciate, embodiments of the anisotropic spinal stabilization rod disclosed herein can be modified or otherwise implemented in many ways without departing from the spirit and scope of the disclosure. Accordingly, this description is to be construed as illustrative only and is for the purpose of teaching those skilled in the art the manner of making and using embodiments of an anisotropic spinal stabilization rod. It is to be understood that the embodiments shown and described herein are to be taken as exemplary. Equivalent elements or materials may be substituted for those illustrated and described herein. Moreover, certain features of the disclosure may be utilized independently of the use of other features, all as would be apparent to one skilled in the art after having the benefit of this description of the disclosure.

1. An implantable rod for stabilizing a portion of a spine, comprising:
two stiff parts; and
one flexible part,
wherein each of the stiff parts and the flexible part has a cylindrical body;
wherein the stiff parts and the flexible part are joined from end to end about a common axis, with the flexible part in between the stiff parts;
wherein the flexible part is made of a composite material;
wherein the composite material comprises a base matrix and fibers; and
wherein the fibers are selectively oriented in one or more directions to achieve desired range of motion of the spine.

2. The implantable rod of claim 1, further comprising a plurality of stiff parts and flexible parts are joined from end to end about the common axis in an alternate pattern.

3. The implantable rod of claim 1, wherein the stiff parts are stiffer than the flexible part by at least one order of magnitude.

4. The implantable rod of claim 1, wherein the stiff parts are made of a composite material with fibers oriented in a direction parallel to the common axis of the implantable rod.

5. The implantable rod of claim 1, wherein the stiff parts are made of a composite material with at least two layers of fibers, wherein fibers in a first layer are oriented in parallel to the common axis of the implantable rod, and wherein fibers in a second layer are oriented perpendicular to the common axis of the implantable rod.

6. The implantable rod of claim 5, wherein fibers in a third layer are oriented circumferentially around the common axis of the implantable rod.

7. The implantable rod of claim 1, wherein strands of the fibers are braided helically and aligned in a direction parallel to the common axis of the implantable rod.

8. The implantable rod of claim 1, wherein the flexible part has a first portion, a second portion, and a third portion, wherein the first portion and the third portion are joined to the stiff parts, wherein fibers in the first portion and the third portion are oriented at a first angle with respect to a horizontal plane and enter the stiff parts at a second angle with respect to the horizontal plane, and wherein fibers from the flexible part, upon entering the stiff parts, are oriented in parallel to the common axis.

9. The implantable rod of claim 8, wherein fibers in the second portion are oriented in a direction parallel to the common axis of the implantable rod.

10. The implantable rod of claim 8, wherein the first angle and the second angle are in the range of about ±28° to 57°.

11. The implantable rod of claim 8, wherein the flexible part has one or more perforated thin discs with angled through holes through which fibers from the second portion are oriented to the first angle.

12. The implantable rod of claim 1, wherein fiber content of the composite material is about 10% to 30% by volume.

13. An implantable rod for stabilizing a portion of a spine, comprising:
a cylindrical body made of a composite material and having two stiff end portions for attachment to a set of bone fasteners and a flexible intermediate portion in between the two stiff end portions;
wherein the composite material comprises a base matrix and fibers;
wherein fibers in the stiff portions are selectively oriented in one or more directions with respect to a horizontal plane, with at least a first set of the fibers in the stiff portions oriented in a first direction that is parallel to the common axis of the implantable rod; and
wherein fibers in the flexible portion are selectively oriented in two or more directions with respect to the horizontal plane.

14. The implantable rod of claim 13, wherein the stiff portions are stiffer than the flexible portion by at least one order of magnitude.

15. The implantable rod of claim 13, wherein a second set of the fibers in the stiff portions is selectively oriented in a second direction that is perpendicular to the common axis of the implantable rod.

16. The implantable rod of claim 15, wherein a third set of the fibers in the stiff portions is selectively oriented circumferentially around the common axis of the implantable rod.

17. The implantable rod of claim 13, wherein the flexible portion includes helically braided fibers aligned in the first direction.

18. The implantable rod of claim 13, wherein the fibers in the flexible portion are oriented in the range of about ±28° to 57°.

19. The implantable rod of claim 13, further comprising one or more perforated thin discs with angled through holes through which fibers are oriented from a first direction to a second direction with respect to the horizontal plane.

20. The implantable rod of claim 13, wherein fiber content of the composite material is about 10% to 30% by volume.

21. An implantable rod for stabilizing a portion of a spine, comprising:
two metal parts; and
one composite part,
wherein each of the metal parts and the composite part has a cylindrical body;
wherein the metal parts and the composite part are joined from end to end about a common axis, with the composite part in between the metal parts;
wherein the composite part is made of a base matrix and fibers;
wherein the metal parts are stiffer than the composite part by at least one order of magnitude; and
wherein the fibers are selectively oriented in one or more directions with respect to a horizontal plane.

22. The implantable rod of claim 21, further comprising a plurality of metal parts and composite parts joined from end to end about the common axis in an alternate pattern.

23. The implantable rod of claim 21, wherein the composite part has a first portion, a second portion, and a third portion, wherein fibers in the first portion and the third portion are oriented in the range of about ±28° to 57° with respect to the horizontal plane.

24. The implantable rod of claim 23, wherein the composite part has one or more perforated thin discs with angled through holes through which fibers from the second portion are oriented from a first direction to a second direction with respect to the horizontal plane.

25. The implantable rod of claim 21, wherein fiber content of the composite part is about 10% to 30% by volume.
